(19) United States
(12) Patent Application Publication
(10) Pub. No.: US 2022/0202647 A1
(43) Pub. Date: Jun. 30, 2022

(54) MASSAGE CONTROL METHOD AND ELECTRONIC DEVICE IMPLEMENTING MASSAGE CONTROL METHOD

(71) Applicants: HONG FU TAI PRECISION ELECTRONS (YANTAI) CO., LTD., Yantai (CN); HON HAI PRECISION INDUSTRY CO., LTD., New Taipei (TW)

(72) Inventors: CHENG-FA CHUNG, New Taipei (TW); NA WANG, Yantai (CN); KUN-LIN HUANG, New Taipei (TW)

(21) Appl. No.: 17/365,072

(22) Filed: Jul. 1, 2021

(30) Foreign Application Priority Data
Dec. 31, 2020 (CN) 202011625333.5

(51) Int. Cl. A61H 39/02; A61H 39/04; A61H 7/00; G16H 10/60; G16H 20/30; G16H 40/60; G05B 13/02

(52) U.S. Cl. CPC A61H 39/02; A61H 39/04; A61H 7/004; G16H 10/60; G16H 20/30; G16H 20/90; G05B 13/024; A61H 2201/5043; A61H 2201/5007; A61H 2201/5064; G16H 40/60

(57) ABSTRACT

A massage control method includes obtaining three-dimensional parameter data of a user, determining acupuncture points, a position of a first part, and a position of a second part of the user according to the three-dimensional parameter data, adjusting a position of a massage device according to the three-dimensional parameter data so that the position of the massage device corresponds to the three-dimensional parameter data, and aligning at least one massage component of the massage device with the acupuncture points according to the positions of the first part and the second part of the user.

### FIELD

The subject matter herein generally relates to massage devices, and more particularly to a massage control method and an electronic device implementing the massage control method.

### BACKGROUND

Generally, a size of a massage chair cannot adapt to users of different sizes. Therefore, massage components of the massage chair are unable to accurately align with acupuncture points of the users of different sizes.

### BRIEF DESCRIPTION OF THE DRAWINGS

Implementations of the present disclosure will now be described, by way of embodiments, with reference to the attached figures.

FIG. 1 is a flowchart of a massage control method according to an embodiment of the present disclosure.

FIG. 2 is a block diagram of a massage control system according to an embodiment of the present disclosure.

FIG. 3 is a block diagram of an electronic device according to an embodiment of the present disclosure.

### DETAILED DESCRIPTION

It will be appreciated that for simplicity and clarity of illustration, where appropriate, reference numerals have been repeated among the different figures to indicate corresponding or analogous elements. Additionally, numerous specific details are set forth in order to provide a thorough understanding of the embodiments described herein. However, it will be understood by those of ordinary skill in the art that the embodiments described herein can be practiced without these specific details. In other instances, methods, procedures and components have not been described in detail so as not to obscure the related relevant feature being described. The drawings are not necessarily to scale and the proportions of certain parts may be exaggerated to better illustrate details and features. The description is not to be considered as limiting the scope of the embodiments described herein.

Several definitions that apply throughout this disclosure will now be presented.

The term “comprising” means “including, but not necessarily limited to”; it specifically indicates open-ended inclusion or membership in a so-described combination, group, series, and the like.

In general, the word “module” as used hereinafter refers to logic embodied in hardware or firmware, or to a collection of software instructions, written in a programming language such as, for example, Java, C, or assembly. One or more software instructions in the modules may be embedded in firmware such as in an erasable-programmable read-only memory (EPROM). It will be appreciated that the modules may comprise connected logic units, such as gates and flip-flops, and may comprise programmable units, such as programmable gate arrays or processors. The modules described herein may be implemented as either software and/or hardware modules and may be stored in any type of computer-readable medium or other computer storage device.

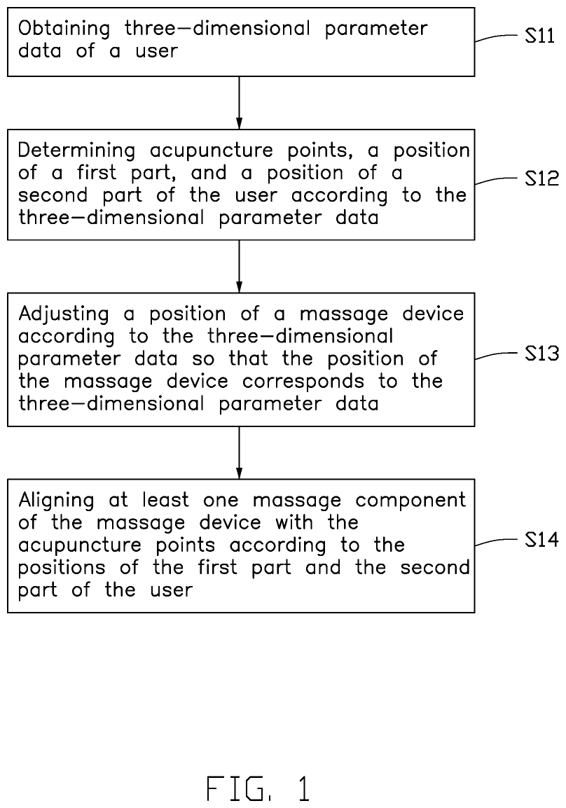

FIG. 1 shows a flowchart of a massage control method according to an embodiment of the present disclosure. According to different needs, the order of blocks in the flowchart can be changed, and some blocks can be omitted or combined.

At block S11, three-dimensional parameter data of a user is obtained.

In one embodiment, obtaining the three-dimensional parameter data of the user includes:

Obtaining a height of the user and a position of at least one body part of the user. The at least one body part may include a hip bone and a spine.

At block S12, acupuncture points, a position of a first part, and a position of a second part of the user are determined according to the three-dimensional parameter data.

In one embodiment, the acupuncture points of the user are determined by:

Confirming positions of the first part and the second part of the user according to the three-dimensional parameter data; and

Determining the acupuncture points of the user according to the positions of the first part and the second part.

In one embodiment, the first part and the second part may be human bones, and the position of the acupuncture points of the user can be determined according to the positions of the first part and the second part. In other embodiments, the first part and the second part may be different organs of the human body.

For example, the first part may be the hip bone, and the second part may be the spine.

Specifically, the positions of different vertebrae in the spine can be determined according to the positions of the hip bone and the spine. For example, positions of the seventh cervical vertebra, first thoracic vertebra, third thoracic vertebra, fifth thoracic vertebra, and sixth thoracic vertebra, the seventh thoracic vertebra, and the ninth thoracic vertebra can be determined.

For example, determining the positions of the acupuncture points of the user according to the positions of the hip bone and the spine may include:

Taking a depression under the spinous process of the seventh cervical vertebra of the user as the Dazhui point of the user;

Taking a depression under the spinous process of the first thoracic vertebra of the user as the Taodao point of the user;

Taking a depression under the spinous process of the third thoracic vertebra of the user as the body column point of the user;

Taking a depression under the spinous process of the fifth thoracic vertebra of the user as the Shendao point of the user;

Taking a depression under the spinous process of the sixth thoracic vertebra of the user as the Lingtai point of the user;

Taking the depression under the spinous process of the seventh thoracic vertebra of the user as the Zhiyang point of the user; and

Taking a depression under the spinous process of the ninth thoracic vertebra of the user as the tendon reduction point of the user.

At block S13, a position of the massage device is adjusted according to the three-dimensional parameter data so that the position of the massage device corresponds to the three-dimensional parameter data. The massage device includes at least one massage component.

In one embodiment, adjusting the position of the massage device according to the three-dimensional parameter data so that the position of the massage device corresponds to the three-dimensional parameter data includes:

Adjusting a movement distance and/or a rotation angle of the at least one massage component of the massage device according to the three-dimensional parameter data.

Specifically, in one embodiment, the movement distance of the at least one massage component in the massage device is adjusted according to a preset ratio according to the height and/or the position of the body part of the user. For example, the corresponding relationship between different heights or height ranges and the position of the massage component can be stored in advance. Therefore, when determining the height of the user, a correct position of the massage component can be determined based on the corresponding relationship, and then the movement distance and movement direction of at least one massage component is adjusted.

For example, when the height of the user is 170 cm, the movement distance of the at least one massage component in the massage device is adjusted according to a distribution state of the acupuncture points for 170 cm.

In one embodiment, the rotation angle of the massage device can be adjusted according to a sitting posture of the user, so that the massage device adapts to the body angle of the user, and the at least one massage component is aligned with the acupuncture points of the user.

At block S14, the at least one massage component of the massage device is aligned with the acupuncture points according to the positions of the first part and the second part of the user.

In one embodiment, aligning the massage component with the acupuncture points according to the positions of the first part and the second part of the user includes:

Determining a first positional relationship between the user and the massage device according to the position of the first part;

Determining a second positional relationship between the acupuncture points and the massage component according to the position of the second part; and

Adjusting the massage device to the acupuncture points according to the first positional relationship and the second positional relationship.

In one embodiment, when the first part is the hip bone and the second part is the spine, aligning the massage component with the acupuncture points according to the positions of the hip bone and the spine of the user includes:

Confirming the positions of the hip bone and the spine of the user according to the three-dimensional parameter data;

Determining the first positional relationship between the user and the massage device according to the position of the hip bone;

Determining the second positional relationship between the acupuncture point and the massage component according to the position of the spine; and

Adjusting the massage component to the acupuncture points according to the first positional relationship and the second positional relationship.

In one embodiment, the method further includes:

Obtaining an acupuncture point distribution image and a massage device image when the user sits on the massage device;

Determining the positions of the acupuncture points from the acupuncture point distribution image;

Determining the position of the massage component from the massage device image;

Calculating a distance difference between the positions of the acupuncture points and the massage component; and

Moving the massage component to the acupuncture points according to the distance difference.

In one embodiment, the massage component is moved to the acupuncture points according to the distance difference between the acupuncture points and the massage component, so as to realize the adjustment and correction of the distance between the massage component and the acupuncture points, thereby improving the massage experience of the user.

In one embodiment, the method further includes:

Displaying the acupuncture point distribution image and the massage device image;

Displaying the positions of the acupuncture points of the acupuncture point distribution image;

Displaying the position of the massage component; and

Receiving an operation command to move the massage component to the acupuncture points.

By displaying the positions of the acupuncture points of the acupuncture point distribution image and the position of the massage component, the user can operate the massage component to move to the acupuncture points.

In one embodiment, the method further includes:

Displaying at least one acupuncture point of the user;

Receiving a first instruction, wherein the first instruction includes information of an acupuncture point to be massaged; and

Determining the acupuncture point to be massaged according to the first instruction.

Specifically, the first instruction may be a touch input.

In one embodiment, the at least one acupuncture point of the user is displayed, the first instruction is a touch input applied on the at least one displayed acupuncture point, and the at least one acupuncture point to be massages is determined according to the first touch instruction.

In one embodiment, the first instruction may be a voice input.

The at least one acupuncture point of the user is displayed, the first instruction is received from the user, and the at least one acupuncture point to be massaged is determined according to the first instruction.

In one embodiment, the method further includes:

Controlling the massage component to massage the determined at least one acupuncture point.

In one embodiment, controlling the massage component to massage the determined at least one acupuncture point includes:

Determining at least one target massage component of the massage components that is opposite to the acupuncture point to be massaged;

Receiving a second instruction, the second instruction including parameters of the target massage component;

Adjusting the parameter of the target massage component according to the second instruction; and

Massaging the determined at least one acupuncture point according to the parameters of the target massage component.

Specifically, the parameters of the target massage component may include, but are not limited to, massage strength, massage mode, massage location, and massage environment.

In one embodiment, the massage device includes a display interface. The display interface includes at least massage component options and parameter setting options. The massage component options are for the user to select the target massage component. The parameter setting options are for the user to set the massage strength, massage mode, massage location, and massage environment.

For example, the point to be massaged may be the Dazhui point. The massage device can determine the target massage component that is opposite to the Dazhui point and receive the second instruction. The second instruction may be a touch command for determining the strength and/or method of the target massage component. The strength and/or method of the target massage component is adjusted according to the second touch instruction.

In summary, the acupuncture points of different users are determined according to the three-dimensional parameter data of the user, so that the massage components are precisely aligned with the acupuncture points of the different users. Furthermore, the acupuncture points and the massage parameters are selected according to the user's instructions, which can improve the accuracy of massaging the acupuncture points and improve the effect of massage.

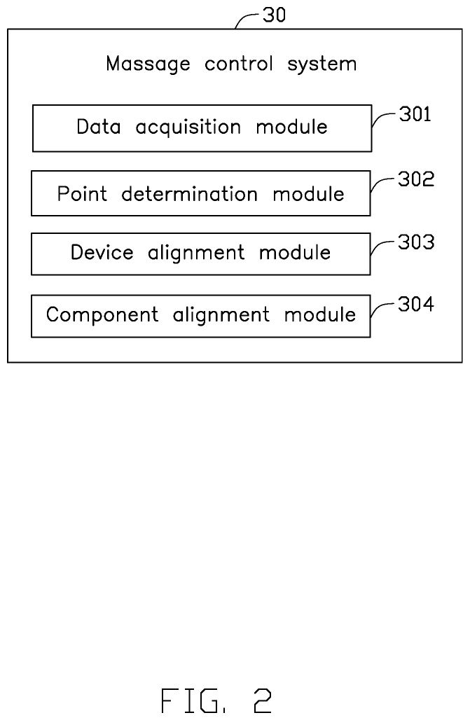

FIG. 2 shows a block diagram of a massage control system 30 according to an embodiment of the present disclosure.

In one embodiment, the massage control system 30 runs in a server. The massage control system 30 may include a plurality of function modules composed of program code segments. The program code segments can be stored in a memory and executed by at least one processor to perform functions of the massage control system 30.

In one embodiment, the function modules of the massage control system 30 may include a data acquisition module 301, a point determination module 302, a device alignment module 303, and a component alignment module 304.

The data acquisition module 301 obtains three-dimensional parameter data of the user.

In one embodiment, the data acquisition module 301 obtains the three-dimensional parameter data as described in block S11, which will not be repeated here.

The point determination module 302 determines acupuncture points, a position of a first part, and a position of a second part of the user according to the three-dimensional parameter data.

In one embodiment, the point determination module 302 determines acupuncture points, a position of a first part, and a position of a second part of the user according to the three-dimensional parameter data as described in block S12, which will not be repeated here.

The device alignment module 303 adjusts a position of a massage device according to the three-dimensional parameter data so that the position of the massage device corresponds to the three-dimensional parameter data. The massage device includes at least one massage component.

In one embodiment, the device alignment module 303 adjusts the position of the massage device as described in block S13, which will not be repeated here.

The component alignment module 304 aligns the at least one massage component of the massage device with the acupuncture points according to the positions of the first part and the second part of the user.

In one embodiment, the component alignment module 304 aligns the at least one massage component of the massage device with the acupuncture points according to the positions of the first part and the second part of the user as described in block S14, which will not be repeated here.

In one embodiment, the massage control system 30 further includes a module for determining acupuncture points to be massaged.

In one embodiment, the acupuncture points to be massaged are determined as described in the above-described massage control method, which will not be repeated here.

In one embodiment, the massage control system 30 further includes a module for controlling the massage device to massage the acupuncture points to be massaged.

In one embodiment, the massage device is controlled to massage the acupuncture points to be massaged as described in the above-described massage control method, which will not be repeated here.

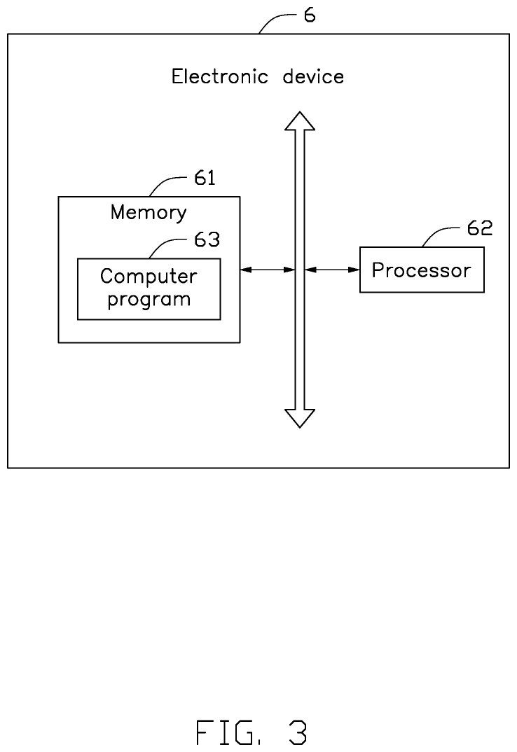

FIG. 3 shows a block diagram of an electronic device 6 according to an embodiment of the present disclosure.

The electronic device 6 includes a memory 61, a processor 62, and a computer program 63 that is stored in the memory 61 and executable by the processor 62. When the computer program 63 is executed, the processor 62 may implement the blocks in the massage control method as shown in FIG. 1. Alternatively, when the computer program is executed, the processor 62 may execute the functions of the modules in the massage control system 30 as shown in FIG. 2.

In one embodiment, the electronic device 6 may include more or less components than those shown in FIG. 3, have a combination of certain components, or have different components. For example, the electronic device 6 may also include an input/output device, a network access device, a bus, and the like.

The processor 62 may be a central processing unit, general-purpose processor, digital signal processor, application specific integrated circuits, field programmable gate array, or other programmable logic device, transistor logic device, discrete hardware component, or the like. The processor 62 can be a microprocessor or a conventional processor. The processor 62 is a control center of the electronic device 6 and connects the parts of the entire electronic device 6 through various interfaces and lines.

The memory 61 may be used to store the computer program 63 and/or modules. The processor 62 runs or executes the computer program and/or modules stored in the memory 61. In addition, the memory 61 may include a non-volatile memory, such as a hard disk, a memory, a plug-in hard disk, a smart memory card, a secure digital card, a flash memory card, at least one disk storage device, flash memory device, etc.

The electronic device 6 may be a computing device such as a desktop computer, a notebook computer, a tablet computer, and a cloud server. The electronic device 6 can interact with the user through a keyboard, a mouse, a remote control, a touch panel, or a voice control device.

The embodiments shown and described above are only examples. Even though numerous characteristics and advantages of the present technology have been set forth in the foregoing description, together with details of the structure and function of the present disclosure, the disclosure is illustrative only, and changes may be made in the detail, including in matters of shape, size and arrangement of the parts within the principles of the present disclosure up to, and including, the full extent established by the broad general meaning of the terms used in the claims.

What is claimed is:

1. A massage control method comprising:
obtaining three-dimensional parameter data of a user;
determining acupuncture points, a position of a first part of the user, and a position of a second part of the user according to the three-dimensional parameter data;
adjusting a position of a massage device according to the three-dimensional parameter data and making the position of the massage device correspond to the three-dimensional parameter data, the massage device comprising at least one massage component; and
aligning the at least one massage component of the massage device with the acupuncture points according to the positions of the first part and the second part of the user.

2. The massage control method of claim 1, further comprising:
displaying at least one acupuncture point of the user;
receiving a first instruction, the first instruction comprising information of an acupuncture point to be massaged; and
determining the acupuncture point to be massaged according to the first instruction.

3. The massage control method of claim 2, further comprising:
controlling the at least one massage component to massage the acupuncture point to be massaged.

4. The massage control method of claim 3, wherein controlling the at least one massage component to massage the acupuncture point to be massaged comprises:
determining, from the at least one massage component, a target massage component that is opposite to the acupuncture point to be massaged;
receiving a second instruction, the second instruction comprising parameters of the target massage component; and
adjusting the parameters of the target massage component according to the second instruction.

5. The massage control method of claim 1, wherein determining the acupuncture points, the position of the first part of the user, and the position of the second part of the user according to the three-dimensional parameter data comprises:
confirming the positions of the first part and the second part of the user according to the three-dimensional parameter data; and
determining the acupuncture points of the user according to the positions of the first part and the second part.

6. The massage control method of claim 1, wherein adjusting the position of the massage device according to the three-dimensional parameter data and making the position of the massage device corresponds to the three-dimensional parameter data comprises:
adjusting a movement distance of the at least one massage component and/or a rotation angle of the massage device according to the three-dimensional parameter data.

7. The massage control method of claim 1, wherein aligning the at least one massage component of the massage device with the acupuncture points according to the positions of the first part and the second part of the user comprises:
determining a first positional relationship between the user and the massage device according to the position of the first part;
determining a second positional relationship between the acupuncture points and the at least one massage component according to the position of the second part; and
aligning the massage device to the acupuncture points according to the first positional relationship and the second positional relationship.

8. An electronic device comprising:
a processor; and
a memory storing a plurality of instructions, which when executed by the processor, cause the processor to:
obtain three-dimensional parameter data of a user;
determine acupuncture points, a position of a first part of the user, and a position of a second part of the user according to the three-dimensional parameter data;
adjust a position of a massage device according to the three-dimensional parameter data and making the position of the massage device corresponds to the three-dimensional parameter data, the massage device comprising at least one massage component; and
align the at least one massage component of the massage device with the acupuncture points according to the positions of the first part and the second part of the user.

9. The electronic device of claim 8, wherein the processor is further configured to:
display at least one acupuncture point of the user;
receive a first instruction, the first instruction comprising information of an acupuncture point to be massaged; and
determine the acupuncture point to be massaged according to the first instruction.

10. The electronic device of claim 9, wherein the processor is further configured to:
control the at least one massage component to massage the acupuncture point to be massaged.

11. The electronic device of claim 10, wherein the processor controls the at least one massage component to massage the acupuncture point to be massaged by:
determining, from the at least one massage component, a target massage component that is opposite to the acupuncture point to be massaged;
receiving a second instruction, the second instruction comprising parameters of the target massage component; and
adjusting the parameters of the target massage component according to the second instruction.

12. The electronic device of claim 8, wherein the processor determines the acupuncture points, the position of the first part, and the position of a second part of the user according to the three-dimensional parameter data by:
confirming the positions of the first part and the second part of the user according to the three-dimensional parameter data; and
determining the acupuncture points of the user according to the positions of the first part and the second part.

13. The electronic device of claim 8, wherein the processor adjusts the position of the massage device according to the three-dimensional parameter data and makes the position of the massage device correspond to the three-dimensional parameter data by:
adjusting a movement distance of the at least one massage component and/or a rotation angle of the massage device according to the three-dimensional parameter data.

14. The electronic device of claim 8, wherein the processor aligns the at least one massage component of the massage device with the acupuncture points according to the positions of the first part and the second part of the user by:
determining a first positional relationship between the user and the massage device according to the position of the first part;
determining a second positional relationship between the acupuncture points and the at least one massage component according to the position of the second part; and
aligning the massage device to the acupuncture points according to the first positional relationship and the second positional relationship.

15. A non-transitory storage medium having stored thereon instructions that, when executed by at least one processor of an electronic device, causes the at least one processor to execute instructions of a massage control method, the massage control method comprising:
obtaining three-dimensional parameter data of a user;
determining acupuncture points, a position of a first part, and a position of a second part of the user according to the three-dimensional parameter data;
adjusting a position of a massage device according to the three-dimensional parameter data so that the position of the massage device corresponds to the three-dimensional parameter data, the massage device comprising at least one massage component; and
aligning the at least one massage component of the massage device with the acupuncture points according to the positions of the first part and the second part of the user.

16. The non-transitory storage medium of claim 15, the massage control method further comprising:
displaying at least one acupuncture point of the user;
receiving a first instruction, the first instruction comprising information of an acupuncture point to be massaged; and
determining the acupuncture point to be massaged according to the first instruction.

17. The non-transitory storage medium of claim 16, the massage control method further comprising:
determining, from the at least one massage component, a target massage component that is opposite to the acupuncture point to be massaged;
receiving a second instruction, the second instruction comprising parameters of the target massage component; and
adjusting the parameters of the target massage component according to the second instruction.

18. The non-transitory storage medium of claim 17, wherein determining acupuncture points, a position of a first part, and a position of a second part of the user according to the three-dimensional parameter data comprises:
confirming the positions of the first part and the second part of the user according to the three-dimensional parameter data; and
determining the acupuncture points of the user according to the positions of the first part and the second part.

19. The non-transitory storage medium of claim 18, wherein adjusting a position of a massage device according to the three-dimensional parameter data so that the position of the massage device corresponds to the three-dimensional parameter data comprises:
adjusting a movement distance of the at least one massage component and/or a rotation angle of the massage device according to the three-dimensional parameter data.

20. The non-transitory storage medium of claim 19, wherein aligning the at least one massage component of the massage device with the acupuncture points according to the positions of the first part and the second part of the user comprises:
determining a first positional relationship between the user and the massage device according to the position of the first part;
determining a second positional relationship between the acupuncture points and the at least one massage component according to the position of the second part; and aligning the massage device to the acupuncture points according to the first positional relationship and the second positional relationship
